SEQUENCE LISTING (12 sequences)

SEQ ID NO: 1 - DNA, 30 bp, Homo sapiens
tccctgcccattcaccgtccatacctggtg

SEQ ID NO: 2 - DNA, 17 bp, Homo sapiens
ccattcaccgtccatac

SEQ ID NO: 3 - DNA, 23 bp, Homo sapiens
ccattcaccgtccatacctggtg

SEQ ID NO: 4 - DNA, 23 bp, Homo sapiens
cctgcccattcaccgtccatacc

SEQ ID NO: 5 - DNA, 23 bp, Homo sapiens
tgcccattcaccgtccatacctg

SEQ ID NO: 6 - DNA, 23 bp, Homo sapiens
gcccattcaccgtccatacctgg

SEQ ID NO: 7 - DNA, 24 bp, Homo sapiens
tccctgcccattcaccgtccatac

SEQ ID NO: 8 - DNA, 25 bp, Homo sapiens
tgcccattcaccgtccatacctggt

SEQ ID NO: 9 - RNA, 17 nt, Homo sapiens
ccauucaccguccauac

SEQ ID NO: 10 - RNA, 17 nt, Artificial Sequence
  interference RNA
guauggacggugaaugg

SEQ ID NO: 11 - DNA, 25 bp, Mus musculus
agcctgctccgattccatacctgat

SEQ ID NO: 12 - DNA, 105 bp, Homo sapiens
ctgcccacactgcccctagtcatggtggcaccctccggggcacggctgggccccttgccccacttacaggcactcctccaggacaggccacatttcatgcaccag

(19) United States
(12) Patent Application Publication
(10) Pub. No.: US 2023/0374506 A1
(43) Pub. Date: Nov. 23, 2023

(54) FOXP3S-PROMOTING MORPHOLINOS

(71) Applicant: THE TRUSTEES OF INDIANA UNIVERSITY, Bloomington, IN (US)

(72) Inventors: Baohua ZHOU, Zionsville, IN (US); Xiongbin LU, Carmel, IN (US); Yujing LI, Carmel, IN (US); Jianguang DU, Carmel, IN (US)

(21) Appl. No.: 18/027,476

(22) PCT Filed: Sep. 9, 2021
(86) PCT No.: PCT/US2021/049606

§ 371 Date: Mar. 21, 2023

Related U.S. Application Data
(60) Provisional application No. 63/083,452, filed on Sep. 25, 2020.

(51) Int. Cl. C12N 15/113; A61P 35/00

(52) U.S. Cl. CPC C12N 15/113; A61P 35/00; A61K 2039/505

(57) ABSTRACT

Disclosed herein are compositions and in vitro and in vivo methods for increasing FOXP3S expression in Tregs in a patient. These compositions and methods are useful for a variety of purposes, including the treatment of cancer.

### CROSS REFERENCE TO RELATED APPLICATIONS

This application claims priority to U.S. Provisional Patent Application No. 63/083,452 filed on Sep. 25, 2020, the disclosure of which is expressly incorporated herein.

### STATEMENT OF US GOVERNMENT SUPPORT

This invention was made with government support under AI085046 and CA203737 awarded by national institutes of health. The government has certain rights in the invention.

### INCORPORATION BY REFERENCES OF MATERIAL SUBMITTED ELECTRONICALLY

Incorporated by reference in its entirety is a computer-readable nucleotide/amino acid sequence listing submitted concurrently herewith and identified as follows: 3 kilobytes ASCII (Text) file named “343969_ST25.txt,” created on Sep. 7, 2021.

### BACKGROUND

Breast cancer is the second most common cancer diagnosed in women in the United States. Currently, the average risk of a woman in the United States developing breast cancer sometime in her life is about 13% (1 in 8 woman) thus about 276,480 new cases of invasive breast cancer will be diagnosed in year 2020. Studies showed that incidence rates of breast cancer were significantly higher in the military population across race and gender, likely due to differences between military woman and general population in reproductive history (age at first birth and the use of contraceptives) and exposure to hazardous chemicals. Despite a decades-long decline in the breast cancer death rate owing to treatment advances, earlier detection through screening and increased awareness, each year over 40,000 patients die from the disease in the U.S.

Regulatory T cells (Tregs) play a central role in maintaining immune system homeostasis and negatively regulate immune-mediated inflammation such as autoimmune diseases, asthma and allergy. However, Tregs also suppress effective immunity against chronic infections and tumors. A systematic review and meta-analysis of 15 published studies comprising 8666 breast cancer patients demonstrated that increased tumor-infiltrating Treg cells in breast cancer are correlated with poorer clinical outcomes including reduced overall survival, tumor malignancy, and metastasis. In contrast, depletion of Tregs with an anti-CD25 antibody (daclizumab) in combination with an experimental cancer vaccine in patients with metastatic breast cancer led to a marked and prolonged decrease in Tregs and robust priming and boosting of CD8⁺and CD4⁺T cells. Consequently, overall survival was improved in this small set of patients. In mouse breast cancer models, depletion or downregulation of Tregs resulted in enhanced anti-tumor immunity and tumor regression.

FOXP3 is a master regulator of Treg development and function. FOXP3 loss of function mutations in both humans and mice result in lethal immunodysregulation, polyendocrinopathy, enteropathy, and X-linked syndrome (IPEX) due to lack of Tregs. While the human FOXP3 gene encodes two major isoforms through mRNA alternative splicing—a long full-length isoform (FOXP3L) and a shorter isoform lacking exon 2 region (FOXP3S), mouse Foxp3 gene only encodes the Foxp3L isoform. Although the FOXP3 exon 2 region (the 2^{nd} protein-coding exon; SEQ ID NO: 12) has been shown to be important in regulating Th17 differentiation, the two FOXP3 isoforms were both effective in directing Treg differentiation and function in vitro under forced overexpression. However, it is often noted that ectopic overexpression of FOXP3L and FOXP3S isoforms in CD4⁺T cells may result in supra-physiological expression levels and are not likely to represent the function of Tregs in vivo. Therefore, it remains an enigma how these two major isoforms differ in determining the functionality and biology of Tregs.

As disclosed herein the relative expression of FOXP3L and FOXP3S isoforms in CD4⁺T cells has been discovered to impact resistance to cancer progression and the efficacy of cancer immunotherapy.

### SUMMARY

The human FOXP3 gene encodes two major isoforms through mRNA alternative splicing—a long full-length isoform (FOXP3L) and a shorter isoform lacking exon 2 region (FOXP3S). However, the mouse Foxp3 gene only encodes the Foxp3L isoform. Thus while FOXP3L is capable of directing Treg development and function, the role of FOXP3S remains elusive. Applicant has discovered that a genetically modified mouse model that only expressed FOXP3S in Tregs conferred resistance to breast cancer progression. Additionally, applicant found that Tregs expressing FOXP3S were unstable and could transdifferentiate to helper-like T cells, thus provide enhanced anti-tumor immunity. Furthermore, patients with higher FOXP3S expression in breast cancer tissues had better overall survival than those had lower FOXP3S expression.

Therefore, in accordance with one embodiment of the present disclosure, a method is provided for enhancing antitumor immunity by promoting FOXP3S expression in Tregs, and more specifically enhancing antitumor immunity against breast cancer. In one embodiment the method comprises administering morpholino antisense oligonucleotides (MOs) to block the inclusion of the exon 2 region during pre-mRNA splicing to shift FOXP3 expression to the FOXP3S isoform. Applicant has verified that this novel MO efficiently suppressed tumor growth in preclinical mouse breast cancer model and patient derived ex vivo breast cancer model and colorectal cancer model.

In accordance with one embodiment, the present invention is directed to inducing regulatory T cells (Tregs) to transdifferentiate into helper-like T cells by modifying intracellular concentrations of FOXP3S relative to FOXP3L. More particularly, in one embodiment the intracellular concentration of FOXP3S is increased relative to FOXP3L in Tregs, optionally resulting in FOXP3S being the predominant FOXP3 isoform present in the modified Tregs. In one embodiment a method is provided for enhancing the expression of FOXP3S in Tregs, and more particularly, inducing Tregs to switch from predominantly FOXP3L expression to predominantly FOXP3S expression. In one embodiment the method comprises the step of decreasing the expression of the FOXP3L relative to FOXP3S expression, and optionally enhancing the expression of FOXP3S.

In accordance with one embodiment the expression of the FOXP3L is decreased by transfecting Tregs with an interference oligomer that inhibits or prevents the expression of FOXP3L. In one embodiment the transfection step takes place in vitro on isolated Tregs or on tumor infiltrating lymphocytes. In one embodiment the interference oligomer targets FOXP3 exon 2 and the Tregs are transfected either in vitro or in vivo. In one embodiment Tregs are transfected with both an interference oligonucleotide that targets exon 2 (present only in FOXP3L) and a nucleic acid that encodes for the FOXP3S isoform, thus simultaneously decreasing the production of FOXP3L and increasing FOXP3S production. In one embodiment the interference oligomer comprises a sequence of nucleobases targeting the following FOXP3 region TCCCTGCCCATTCACCGTCCATACCTGGTG (SEQ ID NO: 1), or a sequence of nucleobases having at least 80%, 85%, 95% or 99% sequence identity with TCCCTGCCCATTCACCGTCCATACCTGGTG (SEQ ID NO: 1), a complement thereof, or a 10, 15, 18, 20, 23 or 25 bp or larger contiguous sequence fragment of SEQ ID NO: 1 or complement thereof. In one embodiment the interference oligomer is a phosphorodiamidate morpholino. In one embodiment the interference oligomer is an interfering RNA comprising a sequence having at least 95% sequence identity with GUAUGGACGGUGAAUGG (SEQ ID NO: 10), optionally wherein the interference oligomer is a phosphorodiamidate morpholino.

In accordance with one embodiment the method of inducing Tregs to transdifferentiate into helper-like T cells can be used to treat cancer. Current treatment options for breast cancer consist of endocrine therapy, chemotherapy, radiation therapy and surgery depending on the subtypes and the stage of breast cancer. The recent advances of immunotherapy has brought in a new treatment algorithm for many types of cancer, raising the enthusiasm for using immunotherapy to treat breast cancer. Normally, Tregs infiltrate breast cancer tissues abundantly thus suppress antitumor immunity. However, applicant has discovered that Tregs modified to express enhanced intracellular concentrations of FOXP3S, relative to FOXP3L transdifferentiated to helper-like T cells, and promote the anti-tumor immune response.

Based upon this finding, FOXP3 targeting oligomers (e.g., iRNAs or morpholino oligos (MO) that target exon 2 of FOX3) can be used to shift cellular production of FOXP3L to FOXP3S expression, and the efficacy of such an approach for providing antitumor activity has been verified in a preclinical mouse model and patient-derived cancer organoid model. Accordingly, these FOXP3S-promoting MOs, or other mechanisms for altering intracellular FOXP3S/FOXP3L ratios, will serve as novel immunotherapies for breast cancer treatment, and the treatment of other solid tumors.

In accordance with one embodiment a human morpholino sequence or other interference nucleic acid is used to induce FOXP3 exon 2 skipping in vivo, and thus shift human FOXP3L isoform to FOXP3S isoform in cells targeted with the interference moieties. In one embodiment morpholinos that induce FOXP3 exon 2 skipping comprise a sequence of TCCCTGCCCATTCACCGTCCATACCTGGTG (SEQ ID NO: 1), or a sequence of nucleobases having at least 85%, 90%, 95% or 99% sequence identity with TCCCTGCCCATTCACCGTCCATACCTGGTG (SEQ ID NO: 1), or a fragment of SEQ ID NO: 1 comprising at least 10, 15, 18, 20, 23 or 25 bp or larger contiguous nucleobases of SEQ ID NO: 1. In one embodiment the interference moiety inducing FOXP3 exon 2 skipping comprises a sequence selected from the group consisting of

(SEQ ID NO: 3)
CCATTCACCGTCCATACCTGGTG;
(SEQ ID NO: 4)
CCTGCCCATTCACCGTCCATACC;
(SEQ ID NO: 5)
TGCCCATTCACCGTCCATACCTG;
(SEQ ID NO: 6)
GCCCATTCACCGTCCATACCTGG;
(SEQ ID NO: 7)
TCCCTGCCCATTCACCGTCCATAC;
and
(SEQ ID NO: 8)
TGCCCATTCACCGTCCATACCTGGT.

In one embodiment a composition comprising one or more iRNAs or morpholinos selected from the group of SEQ ID NOs: 1-8 are used to skip exon 2 of FOX3L and shift cellular production of FOXP3L to FOXP3S expression in targeted cells.

In accordance with one embodiment a human morpholino sequence or other interference nucleic acid is used to block translation of FOXP3L or induce degradation of FOXP3 mRNA containing the exon 2 region thus altering FOXP3S/FOXP3L ratios in cells targeted with the interference moieties. In one embodiment morpholinos or other interference nucleic acids comprise a fragment of CTGCCCACACTGCCCCTAGTCATGGTGGCACCCTCCGGGGCACGGCTGGG CCCCTTGCCCCACTTACAGGCACTCCTCCAGGACAGGCCACATTTCATGCA CCAG (SEQ ID NO: 12) that is at least 10 bp or larger contiguous nucleobases of SEQ ID NO: 12, or a complement thereof, and having at least 85%, 90%, 95% or 99% sequence identity with SEQ ID NO: 12.

### BRIEF DESCRIPTION OF THE DRAWINGS

FIGS. 1A & 1B: Expression of FOXP3S isoform in patients with deletion mutations within exon 2 of FOXP3 gene. FIG. 1A is a table presenting data showing that patients with rare deletion mutations within exon 2 of FOXP3 gene developed autoimmune disease. A flow analysis of FOXP3 expression in patient #2 vs healthy patients is demonstrated in (FIG. 1B). An example of flow cytometry analysis showing CD4 T cells in the peripheral blood mononuclear cells (PBMC) isolated from the patient #2 in FIG. 1A). Regulatory T cells, or Tregs (9.3% of the CD4 T cells) in the patient expressed only the FOXP3S isoform (positively stained by pan-FOXP3 antibody but negative for the exon 2-specific antibody staining).

FIGS. 2A-2C: Foxp3S mice (expressing only the Foxp3S isoform) failed to maintain self-tolerance and developed anti-double stranded DNA (anti-dsDNA) autoantibodies. FIG. 2A is a graph showing ELISA measurement of anti-dsDNA IgG in the serum of WT (Foxp3L) and Foxp3S mice (n=6). FIG. 2B is a graph showing the time course of anti-dsDNA IgG in the serum of Foxp3S mice. Foxp3S mice start to develop anti-dsDNA autoantibody at the age of 40 days. Sera from WT and Foxp3S mice at indicated ages were diluted at 1:500 (n=10 mice). FIG. 2C provides representative images of serum anti-nuclear autoantibodies (ANA) (at 1:160 dilution of serum) detected with fixed mouse 3T3 fibroblast cells. Fluorescent microscopy shows that the serum from Foxp3S mice strongly stains the nuclei of mouse cells (lower right panel), indicating that Foxp3S mice generate ANA. DAPI, or 4′,6-diamidino-2-phenylindole, is a fluorescent dye that binds to DNA thus stains the cell nuclei. Data represent mean±SEM from one of ≥2 independent experiments. **: p<0.01; ***: p<0.001 by two-way ANOVA with Bonferroni post-hoc test.

FIGS. 3A-3C Foxp3S Tregs are unstable. FIG. 3A: Frequencies of Foxp3S Tregs in the thymus and secondary lymphoid organs of heterozygous (Foxp3S/L) female mice suggest that Foxp3S Tregs are unstable. Despite heterozygous female mice have two X chromosomes carrying different alleles (Foxp3L and Foxp3S), each Treg cell only expresses one allele but not both due to random X chromosome inactivation. Accordingly, there are two populations of Tregs (Foxp3L Tregs and Foxp3S Tregs) in the heterozygous female mice. While about 50% Tregs express Foxp3S isoform in the thymus where Tregs develop, the frequency of Foxp3S Tregs in the peripheral lymphoid organs reduce to 20-30%. LN: lymph node. Data represent mean±SD (n=3 mice) from one of 2 independent experiments. FIG. 3B: Lineage tracing mice were used to track the fate of Foxp3L and Foxp3S Tregs. Foxp3-Cremediated excision of the floxed STOP cassette results in constitutive, heritable expression of YFP, even for unstable Treg cells that have lost Foxp3 expression. The data presented in FIG. 3C indicate that Foxp3S Tregs tend to lose Foxp3 expression (in Dim or Neg populations) in comparison with Foxp3L Tregs. Thus, greatly reduced frequency of Foxp3S Tregs in the peripheral lymphoid organs seen in FIG. 3A is most likely due to the instability of Foxp3S Tregs. FL: Foxp3L Tregs; FS: Foxp3S Tregs. Data represent mean±SEM (n=3 mice) *: p<0.05; ***: p<0.001 by two-tailed t test.

FIG. 4A-4C: Foxp3S Tregs, but not Foxp3L Tregs, are capable of helping B cells to produce autoantibodies. Foxp3S and Foxp3L Tregs were purified and transferred into T cell receptor knockout (Tcrb KO) mice as indicated (FIG. 4A). The recipient mice were sacrificed 3 months after Treg cell transfer. ELISA quantification of anti-dsDNA IgG in the serum of the recipient mice transferred with Foxp3L (FL), Foxp3S (FS), or both (FL+FS) Tregs is shown in FIG. 4B. Representative images of anti-nuclear autoantibodies in the serum of the recipient mice, detected with fixed mouse 3T3 fibroblast cells is provided in FIG. 4C. Since the Tcrb KO recipient mice themselves do not have T helper cells and Tregs, the anti-dsDNA and anti-nuclear autoantibody signals seen in the Foxp3S Treg-transferred Tcrb KO recipient mice indicate that Foxp3S Tregs are able to help B cells to produce antibodies, a feature similar to T helper cells, while Foxp3L Tregs do not exhibit this activity. Moreover, simultaneously transferred Foxp3L Tregs in the group of FL+FS Tregs suppress the autoantibody production induced by Foxp3S Tregs. Data represent mean±SEM (n=6 mice) *: p<0.05; ***: p<0.001 by two-way ANOVA with Bonferroni post-hoc test. FS: Foxp3S; FL: Foxp3L.

FIGS. 5A-5D: Foxp3S isoform protects mice from tumor growth of different types. Wildtype C57BL/6 mice (expressing only the Foxp3L isoform) or homozygous Foxp3S mice (expressing only the Foxp3S isoform) were inoculated with various types of mouse cancer cells and tumor growth was monitored. Graphs (FIGS. 5A-5D) present tumor growth in mice injected with 1×10⁶ EO771 triple-negative breast cancer (TNBC) cells (FIG. 5A); mice injected with 0.5×10⁶ Lewis lung carcinoma (LLC) cells (FIG. 5B); mice injected with 5×10⁴ B16 melanoma cells (FIG. 5C); and mice injected with 5×10⁴ MC38 colon adenocarcinoma cells (FIG. 5D). ****, P<0.0001 by two-way ANOVA with Tukey's test for correction (n=5 mice per group).

FIGS. 6A-6D: Functional characterization of T cells in EO771 tumors from WT and Foxp3S mice. FIG. 6A shows the distribution of tumor-infiltrating lymphocytes in EO771 tumors from WT and Foxp3S mice. Single cell RNA sequencing was performed to analyze tumor infiltrating lymphocytes harvested from C57BL/6 WT (n=3) or Foxp3S mice (n=4) on 10 days after EO771 cells inoculation. *, P<0.05; ***, P<0.001. ****, P<0.0001 by two-way ANOVA with Tukey's test for correction. FIG. 6B shows the single cell T cell receptor (TCR) profiling analysis of clonal expansion of T cells in the E0771 TNBC tumors from WT (Hatched column) or Foxp3S mice. FIGS. 6C & 6D show the antibody mediated depletion of CD4 T cells, CD8 T cells, B cells (α-CD19 & CD20), and NK cells in WT (FIG. 6C) and Foxp3S mice (FIG. 6D). While depleting CD4 T cells, B cells, or NK cells had no effect on the Foxp3S isoform's ability to prevent tumor growth, depletion of CD8 T cells rescued tumor growth. The results suggest that the tumor-suppressive function of Foxp3S Tregs is dependent on CD8 T cells.

FIGS. 7A & 7B FOXP3S mRNA expression in triple-negative breast cancer (TNBC) patient tissues is positively correlated with overall survival and antitumor immunity. FIG. 7A presents Kaplan-Meier survival curves (overall survival) for TNBC patients with high expression (>median) or low expression (≤median) of FOXP3S mRNA in The Cancer Genome Atlas (TCGA) cohort. P values were determined by two-sided log-rank test. HR: Hazard Ratio. FIG. 7B shows the positive correlation of FOXP3S mRNA expression with computed cytotoxic lymphocytes proportion in human TNBC tumor tissues. Statistical significance was determined by Mann-Whitney-Wilcoxon test.

FIGS. 8A-8F. Efficacy of intratumoral Foxp3 exon 2 targeting morpholino (E2 MO; comprising the sequence AGCCTGCTCCGATTCCATACCTGAT SEQ ID NO: 11) in TNBC treatment. FIG. 8A shows the experimental design. 1×10⁶ EO771 TNBC cells were orthotopically injected into the 4th mammary fat pad on both sides of the mice. On days 12, 14, 16 and 18, 30 nmol/mouse of E2 MO or control MO was injected intratumorally into the tumors at the left side while the tumors on the right side were not injected and used as internal controls. FIGS. 8B & 8C presents data on breast cancer growth on the treated side (FIG. 8B) and untreated side (FIG. 8C). Intratumoral injection of the E2 MO not only significantly suppressed the left-side (treated side) tumor growth but also slowed down the right-side (untreated side) tumor growth. ***: p<0.001 by two-way ANOVA with Tukey's test for correction. FIG. 8D: Intratumoral E2 MO treatment converted Foxp3L Tregs to Foxp3S Tregs. While no Foxp3S Tregs were seen in control group, ˜20% Tregs in the E2 MO-injected tumors expressed the Foxp3S isoform and ˜6% Tregs in the tumors on the untreated side also expressed the Foxp3S isoform. FIGS. 8E & 8F: Significantly higher proportion of CD4 (FIG. 8E) and CD8 (FIG. 8F) T cells in the E2 MO-injected tumors expressed IFN-γ.

FIGS. 9A-9C: Efficacy of the mouse Foxp3 exon 2 targeting morpholino (E2 MO; comprising the sequence AGCCTGCTCCGATTCCATACCTGAT SEQ ID NO: 11) in adoptive cell transfer (ACT) therapy. All mice were inoculated with 1×10⁶ EO771 TNBC cells before receiving treatment described below. FIG. 9A: Heterozygous Foxp3S/L female mice showed partial resistance to EO771 tumor growth despite only 20-30% Tregs in the periphery expressing the Foxp3S isoform (cf. FIG. 3B). Intraperitoneal injection of anti-mouse PD-1 (10 mg/kg) antibody (twice weekly between day 12 and day 24 post tumor inoculation) further enhanced antitumor ability of Foxp3S/L mice, but not WT mice. The effect of Foxp3S Tregs on antitumor immunity is demonstrated in FIG. 9B. 2×10⁶ Tregs isolated from donor WT or Foxp3S mice were injected intratumorally to the recipient C57BL/6 WT mice on day 12. FIG. 9C shows the effects of adoptive transfer of T cells treated with E2 MO enhanced antitumor immunity. Total T cells from the draining lymph nodes of tumor-bearing C57BL/6 CD45.2 WT mice were extracted and expanded with 1 μM control (Ctrl) or E2 MO in vitro for 7 days. BL/6 CD45.1 mice were inoculated with 1×10⁶ EO771. On the day 10 post tumor inoculations, BL/6 CD45.1 mice received 5 Gy total body irradiation, followed by adoptive transfer of 2×10⁶ expanded T cells per mouse. Mice were administered with 4×10⁴ IU (International Unit) of IL-2 daily for the first four days following T cell transfer. Combination of anti-mouse PD-1 (10 mg/kg, administered 2 days after adoptive cell transfer and thereafter twice weekly for a total of 3 doses) further enhanced antitumor immunity. **, P<0.01; ****, P<0.0001 by two-way ANOVA with Tukey's test for correction (n=5 mice per group).

FIGS. 10A & 10B. Effects of the human exon 2 targeting morpholino on human Tregs. Peripheral blood mononuclear cells were obtained from healthy donors and cultured in vitro with 1 μM E2 MO (comprising human morpholino oligo TGCCCATTCACCGTCCATACCTGGT (SEQ ID NO: 8)) for 3 weeks. As shown in FIG. 10A, the morpholino efficiently shifted FOXP3 expression in Tregs to the short isoform lacking exon 2 (negative for FOXP3 Exon 2). E2 MO treatment greatly reduced CD25 (critical for Treg development, function and stability) and CTLA-4 (a suppressive molecule on Tregs) expression on Tregs, but upregulated CD4OL (a co-stimulatory molecule expressed on T helper cells) expression. As shown in FIG. 10B, E2 MO-treated Tregs express more IFN-γ (increased percentage and mean fluorescent intensity) in response to 20 ng/ml IL-12 stimulation. These data suggest that human Tregs cultured in E2 MO for three weeks can become helper-like T cells (low suppressive molecule and high co-stimulatory molecules).

FIGS. 11A-11H: Enhanced killing of patient-derived breast and colon cancer organoid cells by autologous TILs expanded in the presence of human exon 2 targeting morpholino. Tumor infiltration lymphocytes (TIL) were expanded in vitro for 7 days in the presence or absence of 1 μM exon 2 targeting morpholino (E2 MO comprising human morpholino oligo TGCCCATTCACCGTCCATACCTGGT (SEQ ID NO: 8)) that enhanced FOXPS isoform expression. Patient-derived organoids were either cultured alone (No TIL), with expanded autologous TILs (+TIL), or with autologous TILs expanded in the presence of E2 MO (+MO TIL). The results of tested breast cancer samples are shown in FIGS. 11A-11D, with 3 out of 4 samples: UH2103-31 (FIG. 11B), UH2011-16 (FIG. 11C), and UH2105-32 (FIG. 11D) exhibiting enhanced killing of tumor organoid cells by E2 MO treated T cells. The results of tested colon cancer cell are shown in FIG. 11E-11H, with 3 out of 4 samples: EH2103-02 (FIG. 11F), UH2103-37 (FIG. 11G), and EH2105-01 (FIG. 11H) exhibiting enhanced killing of tumor organoid cells by E2 MO treated T cells. The organoid size was measured as project area (nm²) using Image J software and presented as mean ±SD from different patients. *, p<0.05; **, p<0.01; ***, p<0.001; ****, p<0.0001; n.s., not significant by one-way ANOVA with Turkey's test for correction.

### DETAILED DESCRIPTION

### Definitions

In describing and claiming the invention, the following terminology will be used in accordance with the definitions set forth below.

The term “about” as used herein means greater or lesser than the value or range of values stated by 10 percent but is not intended to limit any value or range of values to only this broader definition. Each value or range of values preceded by the term “about” is also intended to encompass the embodiment of the stated absolute value or range of values.

As used herein, the term “purified” and like terms relate to the isolation of a molecule or compound in a form that is substantially free of contaminants normally associated with the molecule or compound in a native or natural environment. As used herein, the term “purified” does not require absolute purity; rather, it is intended as a relative definition.

The term “isolated” requires that the referenced material be removed from its original environment (e.g., the natural environment if it is naturally occurring). For example, a naturally-occurring polynucleotide present in a living animal is not isolated, but the same polynucleotide, separated from some or all of the coexisting materials in the natural system, is isolated.

Tissue nanotransfection (TNT) is an electroporation-based technique capable of delivering nucleic acid sequences and proteins into the cytosol of cells at nanoscale. More particularly, TNT uses a highly intense and focused electric field through arrayed nanochannels, which benignly nanoporates the juxtaposing tissue cell members, and electrophoretically drives cargo (e.g., nucleic acids or proteins) into the cells.

As used herein a “control element” or “regulatory sequence” are non-translated regions of a functional gene, including enhancers, promoters, 5′ and 3′ untranslated regions, which interact with host cellular proteins to carry out transcription and translation. Such elements may vary in their strength and specificity. “Eukaryotic regulatory sequences” are non-translated regions of a functional gene, including enhancers, promoters, 5′ and 3′ untranslated regions, which interact with host cellular proteins of a eukaryotic cell to carry out transcription and translation in a eukaryotic cell including mammalian cells.

As used herein a “promoter” is a sequence or sequences of DNA that function when in a relatively fixed location in regard to the transcription start site of a gene. A “promoter” contains core elements required for basic interaction of RNA polymerase and transcription factors and can contain upstream elements and response elements.

As used herein an “enhancer” is a sequence of DNA that functions independent of distance from the transcription start site and can be either 5′ or 3′ to the transcription unit. Furthermore, enhancers can be within an intron as well as within the coding sequence itself. They are usually between 10 and 300 bp in length, and they function in cis Enhancers function to increase transcription from nearby promoters. Enhancers, like promoters, also often contain response elements that mediate the regulation of transcription. Enhancers often determine the regulation of expression.

The term “identity” as used herein relates to the similarity between two or more sequences. Identity is measured by dividing the number of identical residues by the total number of residues and multiplying the product by 100 to achieve a percentage. Thus, two copies of exactly the same sequence have 100% identity, whereas two sequences that have amino acid or nucleotide deletions, additions, or substitutions relative to one another have a lower degree of identity. Those skilled in the art will recognize that several computer programs, such as those that employ algorithms such as BLAST (Basic Local Alignment Search Tool, Altschul et al. (1993) J. Mol. Biol. 215:403-410) are available for determining sequence identity.

The term “stringent conditions” is functionally defined with regard to the hybridization of a first nucleic acid to a second target nucleic acid (i.e., to a particular nucleic-acid sequence of interest) by the specific hybridization procedure discussed in Sambrook et al., 1989, at 9.52-9.55. See also, Sambrook et al., 1989 at 9.47-9.52 and 9.56-9.58. In one embodiment stringent conditions include conducting hybridization of a first and second nucleic acid using a solution comprising about 0.02 M to about 0.15 M NaCl at temperatures of about 50° C. to about 70° C., followed by washing with a high-stringency wash buffer (0.2X SSC, 0.1% SDS, 65° C.).

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

As used herein, the term “treating” includes alleviation of the symptoms associated with a specific disorder or condition and/or preventing or eliminating said symptoms.

As used herein an “effective” amount or a “therapeutically effective amount” of a drug refers to a nontoxic but enough of the drug to provide the desired effect. The amount that is “effective” will vary from subject to subject or even within a subject overtime, depending on the age and general condition of the individual, mode of administration, and the like. Thus, it is not always possible to specify an exact “effective amount.” However, an appropriate “effective” amount in any individual case may be determined by one of ordinary skill in the art using routine experimentation.

As used herein the term “patient” without further designation is intended to encompass any warm blooded vertebrate domesticated animal (including for example, but not limited to livestock, horses, cats, dogs and other pets) and humans.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

The term “inhibit” defines a decrease in an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

The term “vector” or “construct” designates a nucleic acid sequence capable of transporting into a cell another nucleic acid to which the vector sequence has been linked. The term “expression vector” includes any vector, (e.g., a plasmid, cosmid or phage chromosome) containing a gene construct in a form suitable for expression by a cell (e.g., linked to a transcriptional control element). “Plasmid” and “vector” are used interchangeably, as a plasmid is a commonly used form of vector. Moreover, the invention is intended to include other vectors which serve equivalent functions.

The term “operably linked to” refers to the functional relationship of a nucleic acid with another nucleic acid sequence. Promoters, enhancers, transcriptional and translational stop sites, and other signal sequences are examples of nucleic acid sequences that can operably linked to other sequences. For example, operable linkage of DNA to a transcriptional control element refers to the physical and functional relationship between the DNA and promoter such that the transcription of such DNA is initiated from the promoter by an RNA polymerase that specifically recognizes, binds to and transcribes the DNA.

As used herein the term “transfection” or “transfect” defines a process where a nucleic acid sequence, or a mimetic analog thereof, is internalized by eukaryotic cells, passing across the cell membrane and into the cytoplasm. Transfection can include passive mean (e.g. receptor mediated uptake, passage through cell pores) as well as external assisted means including by physical (e.g., electroporation), chemical (e.g., cationic lipid or calcium phosphate), or molecular modification (modifications to enhance cellular delivery or in vivo stability) mechanisms.

As used herein “interference oligomer” is any nucleic acid oligonucleotide or analog thereof that participates in post-transcriptional gene regulation such as silencing and splicing. Examples of interference oligomers includes, but is not limited to, phosphorodiamidate morpholino, double stranded RNA (dsRNA), small interfering RNA (siRNA), and microRNA (miRNA) that are comprised of sense and/or antisense strands.

As used herein the term “phosphorodiamidate morpholino” designates a DNA/RNA analog wherein the phosphodiester backbone of the DNA/RNA is substituted with a backbone of morpholine rings connected by phosphorodiamidate linkages.

As used herein the term “FOXP3S-promoting morpholino” designates a phosphorodiamidate morpholino that upon introduction to a cell will increase intracellular concentrations of FOXP3S relative to FOXP3L. In one embodiment the FOXP3S-promoting phosphorodiamidate morpholino is an oligonucleotide that targets and induces the exclusion of the FOXP3 exon 2 from FOXP3 mRNA thus promoting FOXP3S expression.

As used herein the term “transdifferentiate” defines an artificial process in which one mature somatic cell is transformed into another mature somatic cell without undergoing an intermediate pluripotent state or progenitor cell type.

### Embodiments

The human FOXP3 gene encodes two major isoforms through mRNA alternative splicing—a long full-length isoform (FOXP3L) and a shorter isoform lacking exon 2 region (FOXP3S). However, the mouse Foxp3 gene only encodes the Foxp3L isoform. Thus while the FOXP3L promotes Treg development and function, the role of FOXP3S remains elusive. Applicant has discovered that a genetically modified mouse model that only expressed FOXP3S in Tregs conferred resistance to breast cancer and colon cancer progression. Additionally, applicant found that Tregs expressing FOXP3S were unstable and could transdifferentiate to helper-like T cells, thus provide enhanced anti-tumor immunity. Furthermore, breast cancer patients with higher FOXP3S transcripts in tumor tissues had better overall survival.

Therefore, in accordance with one embodiment of the present disclosure, a method is provided for enhancing antitumor immunity by promoting FOXP3S expression in Tregs, and more specifically enhancing antitumor immunity against breast cancer. In one embodiment the method comprises administering morpholino antisense oligonucleotides (MOs) to block the inclusion of exon 2 region during pre-mRNA splicing to shift FOXP3 expression to the FOXP3S isoform.

In accordance with one embodiment, the present disclosure is directed to inducing regulatory T cells (Tregs) to transdifferentiate to helper-like T cells by modifying intracellular concentrations of FOXP3S relative to FOXP3L. More particularly, as disclosed herein, a method is provided for inducing regulatory T cells (Tregs) to shift from FOXP3L production to FOXP3S production. In one embodiment, the method comprises the step of decreasing the expression of the FOXP3L isoform relative to FOXP3S expression. In one embodiment the expression of the FOXP3L isoform is decreased by transfecting said Tregs with a FOXP3S-promoting interference oligomer, in an amount effective to shift expression of FOXP3 in said Tregs to the FOXP3S isoform.

The method of inducing Tregs to shift expression of FOXP3 to the FOXP3S isoform can be conducted in vitro or in vivo. In one embodiment regulatory T cells (Tregs) or tumor infiltrating lymphocytes are recovered from a patient, induced to transdifferentiate into helper-like T cells in vitro (by increasing cellular FOXP3S production relative to FOXP3L production) and then are returned to the patient. In one embodiment regulatory T cells (Tregs) are recovered from a patient and contacted with one or more interference oligomers that target FOXP3 under conditions wherein the interference oligomers are taken up by the cell and inhibit and/or silence FOXP3 expression. Silencing or inhibiting the FOXP3L isoform expression with a morpholino or iRNA will induce tumor reactive Tregs to become helpers-like T cells. Such in vitro induced T helper cells can be reintroduced into the patient either alone or in conjunction with other standard anticancer therapies, including for example, co-administration with in vitro expanded tumor infiltrating lymphocytes.

In one embodiment Tregs are transfected in vivo with nucleic acid sequences, or nucleic acid sequence analogs, that target exon 2 of FOXP3 in a patient in need of enhanced immunotherapeutic efficacy. Interference oligomers can be placed in contact with Tregs in vivo under conditions where Tregs take up the exon 2 targeting interference oligomers and increase intracellular concentrations of FOXP3S relative to FOXP3L, optionally by shifting expression from FOXP3L to FOXP3S expression in the Tregs contacted with the interference oligomers. In one embodiment oligomers suitable for targeting exon 2 of FOXP3 comprise a sequence that specifically binds to the sequence CCATTCACCGTCCATAC (SEQ ID NO: 2) or CCAUUCACCGUCCAUAC (SEQ ID NO: 9) or a sequence having at least 90%, 95% or 99% sequence identity with SEQ ID NO: 2 or SEQ ID NO: 9. In one embodiment interference oligomers suitable for targeting exon 2 of FOXP3 comprise a sequence that binds to the sequence CCATTCACCGTCCATAC (SEQ ID NO: 2) or CCAUUCACCGUCCAUAC (SEQ ID NO: 9) under stringent hybridization conditions. In accordance with one embodiment an interference oligomers suitable for excluding exon 2 of FOXP3 comprises a sequence that binds to the sequence selected from the group of

(SEQ ID NO: 3)
CCATTCACCGTCCATACCTGGTG,
(SEQ ID NO: 4)
CCTGCCCATTCACCGTCCATACC,
(SEQ ID NO: 5)
TGCCCATTCACCGTCCATACCTG,
(SEQ ID NO: 6)
GCCCATTCACCGTCCATACCTGG,
(SEQ ID NO: 7)
TCCCTGCCCATTCACCGTCCATAC,
and
(SEQ ID NO: 8)
TGCCCATTCACCGTCCATACCTGGT.

In one embodiment a method is provided for enhancing the expression of FOXP3S in Tregs relative to the expression of FOXP3L, and more particularly, inducing Tregs to switch from expressing FOXP3L to predominantly expressing FOXP3S. In one embodiment the method comprises the step of decreasing the expression of the FOXP3L relative to FOXP3S expression, and optionally enhancing the expression of FOXP3S. In one embodiment the step of decreasing the expression of the FOXP3L relative to FOXP3S expression is conducted by introducing interference oligomers into the cytoplasm of Tregs wherein the interference oligomers specifically interfere with the expression of FOXP3L, optionally by targeting the sequence of exon 2. In one embodiment the expression of FOXP3S is enhanced, optionally in conjunction with interfering with FOXP3L expression, optionally by introducing into the Tregs nucleic acid sequences encoding FOXP3S.

In accordance with one embodiment the expression of the FOXP3L is decreased and the expression of FOXP3S is increased by transfecting Tregs with an interference oligomer that targets FOXP3 exon 2 and comprises a sequence that binds to
i) nucleobases TCCCTGCCCATTCACCGTCCATACCTGGTG (SEQ ID NO: 1) or a complete complement thereof, or
ii) a sequence of nucleobases having at least 90%, 95% or 99% sequence identity with TCCCTGCCCATTCACCGTCCATACCTGGTG or a complete complement thereof, or
iii) a contiguous 10, 15, 17, 20 or 25 bp or longer fragment sequence of SEQ ID NO: 1 or its complement thereof, or
iv) any combination of i), ii) or iii). In one embodiment the interference oligomer is an oligonucleotide, including for example an interference RNA, including for example a small interfering RNA (siRNA), or microRNA (miRNA). In one embodiment the interference oligomer is a modified DNA wherein the phosphodiester backbone of the native DNA has been replaced with a non-ionic mimetic. In one embodiment the interference oligomer is a phosphorodiamidate morpholino. In one embodiment the interference oligomer is an interfering RNA comprising a sequence having at least 95% sequence identity with GUAUGGACGGUGAAUGG or its complement thereof, optionally wherein the interfering oligomer comprises nucleobases GUAUGGACGGUGAAUGG formed as an RNA or phosphorodiamidate morpholino oligomer.

In accordance with one embodiment the expression of the FOXP3L is decreased by transfecting Tregs with an interference oligomer that targets FOXP3 exon 2 and comprises a sequence that binds to
i) nucleobases CTGCCCACACTGCCCCTAGTCATGGTGGCACCCTCCGGGGCACGGCTGGG CCCCTTGCCCCACTTACAGGCACTCCTCCAGGACAGGCCACATTTCATGCA CCAG (SEQ ID NO: 12) or a complete complement thereof, or
ii) a contiguous 10, 15, 17, 20 or 25 bp or longer fragment sequence of SEQ ID NO: 12 or its complement thereof. In one embodiment the interference oligomer is an oligonucleotide, including for example an interference RNA, including for example a small interfering RNA (siRNA), or microRNA (miRNA). In one embodiment the interference oligomer is a phosphorodiamidate morpholino.

In accordance with one embodiment Tregs are induced to transdifferentiate into helper-like T-cells as a therapeutic approach to treating cancer. Current treatment options for breast cancer consist of endocrine therapy, chemotherapy, radiation therapy and surgery depending on the subtypes and the stage of breast cancer. The recent advances of immunotherapy have brought in a new treatment algorithm for many types of cancer, raising the enthusiasm for using immunotherapy to treat breast cancer. Normally, Tregs infiltrate breast cancer tissues abundantly, and thus suppress antitumor immunity. However, applicant has discovered that Tregs expressing FOXP3S will transdifferentiated into helper-like T cells, and thus promote the anti-tumor immune response.

Based upon this finding, FOXP3 targeting oligomers (e.g., iRNAs or morpholino oligos (MO)) can be used to shift cellular production of FOXP3L to FOXP3S expression, and the efficacy of such an approach for providing antitumor activity has been verified in a preclinical mouse models and patient derived organoid models. Accordingly, these FOXP3S-promoting MOs, or other mechanisms for altering intracellular FOXP3S/ FOXP3L ratios, will serve as novel immunotherapies for breast cancer treatment, and the treatment of other solid tumors.

In one embodiment the method of treating cancer in a patient comprises the step of increasing the relative concentration of FOXP3S expressing regulatory T cells relative to FOXP3L expressing regulatory T cells. In accordance with one embodiment a method for treating a patient having a solid tumor is provided wherein the number of Tregs expressing FOXP3S is increased in said patient, optionally wherein the increased number of Tregs expressing FOXP3S is localized to the site of the tumor. In one embodiment the number of Tregs expressing FOXP3S is increased due to the introduction of Tregs expressing FOXP3S to said patient. In one embodiment T cells are recovered form a patient, and the T cells are treated to induce regulatory T cells to shift from FOXP3L isoform to FOXP3S expression before the T cells are reintroduced into the patient. In one embodiment the in vitro induced cells are infused intravenously into the patient or injected into the tissues harboring the solid tumor.

In one embodiment a method of treating solid tumors, including breast cancer comprises a step of inducing regulatory T cells (Tregs) to differentiate into tumor-reactive helper-like T cells by transfecting said Tregs with an interference oligomer in an amount effective to increase FOXP3S expression in said Tregs. In one embodiment Tregs are transfected with one or more interference oligomers that interfere with FOXP3L expression and/or nucleic acids sequences that encode for the FOXP3S isoform. In one embodiment the Tregs are transfected with an interference oligomer that targets exon 2 of FOXP3. In one embodiment the Tregs are transfected in vivo using standard techniques, including for example tissue nanotransfection. In one embodiment interference oligomer is introduced into the cytosol of Tregs wherein the oligomer is a FOXP3S-promoting morpholino.

In one embodiment the method of treating cancer by inducing the transdifferentiation of Tregs to helper-like T cells is conducted in conjunction with other known immunological or other cancer treatment therapies. In one embodiment the method of treating cancer disclosed herein further comprises the step of administering to said patient an immune checkpoint blockade antibody.

### Additional Embodiments

In accordance with embodiment 1, a method for altering regulatory T cells

(Tregs) activity is provided wherein said method comprises the step of modifying intracellular concentrations of FOXP3 isoforms FOXP3L and FOXP3S in said Tregs, wherein the amount of the FOXP3S isoform is increased relative to the FOXP3L isoform.

In accordance with embodiment 2, the method of embodiment 1 is provided wherein the expression of the FOXP3L is inhibited relative to FOXP3S by transfecting Tregs with an interference oligomer that targets FOXP3L, optionally by excluding exon 2 of FOXP3.

In accordance with embodiment 3, the method of embodiment 1 or 2 is provided wherein the expression of the FOXP3S is enhanced relative to FOXP3L by transfecting Tregs with a nucleic acid encoding for the FOXP3S isoform.

In accordance with embodiment 4, the method of any one of embodiments 1-3 is provided wherein said Tregs are transfected with an amount of said interference oligomer sufficient to induce the isolated Tregs to transdifferentiate into helper-like T cells.

In accordance with embodiment 5, the method of any one of embodiments 1-4 is provided wherein the relative expression of the FOXP3L isoform is decreased by transfecting said Tregs with a FOXP3L targeting interference oligomer comprising
i) a nucleobase sequence having at least 85% sequence identity with SEQ ID NO: 1, or
ii) at least a contiguous 10 nucleobase fragment of SEQ ID NO: 1; or
iii) a complement of i) or ii).

In accordance with embodiment 6, the method of any one of embodiments 1-5 is provided wherein said targeting interference oligomer comprises the nucleobase sequence of TGCCCATTCACCGTCCATACCTGGT (SEQ ID NO: 8), or a complement thereof.

In accordance with embodiment 6.5, the method of any one of embodiments 1-10 6 is provided wherein the expression of FOXP3L is decreased by transfecting Tregs with an interference oligomer that targets FOXP3 exon 2 and comprises a sequence that binds to
i) nucleobases CTGCCCACACTGCCCCTAGTCATGGTGGCACCCTCCGGGGCACGGCTGGG CCCCTTGCCCCACTTACAGGCACTCCTCCAGGACAGGCCACATTTCATGCA CCAG (SEQ ID NO: 12) or a complete complement thereof, or
ii) a contiguous 10, 15, 17, 20 or 25 bp or longer fragment sequence of SEQ ID NO: 12 or its complement thereof, optionally wherein the interference oligomer is an oligonucleotide, including for example an interference RNA, including for example a small interfering RNA (siRNA), or microRNA (miRNA), optionally wherein the interference oligomer is a phosphorodiamidate morpholino.

In accordance with embodiment 7, the method of any one of embodiments 1-6.5 is provided wherein the relative expression of the FOXP3L isoform is decreased by transfecting said Tregs with a FOXP3L targeting interference oligomer that specifically binds to a sequence comprising CCATTCACCGTCCATAC (SEQ ID NO: 2) or CCAUUCACCGUCCAUAC (SEQ ID NO: 9).

In accordance with embodiment 8, the method of any one of embodiments 1-7 is provided wherein the interference oligomer specifically binds to a sequence selected from the group consisting of
CCATTCACCGTCCATACCTGGTG (SEQ ID NO: 2),
CCTGCCCATTCACCGTCCATACC (SEQ ID NO: 4),
TGCCCATTCACCGTCCATACCTG (SEQ ID NO: 5),
GCCCATTCACCGTCCATACCTGG (SEQ ID NO: 6),
TCCCTGCCCATTCACCGTCCATAC (SEQ ID NO: 7)
TGCCCATTCACCGTCCATACCTGGT (SEQ ID NO: 8) and
TCCCTGCCCATTCACCGTCCATACCTGGTG (SEQ ID NO: 1) or its RNA counterpart.

In accordance with embodiment 9, the method of any one of embodiments 1-8 is provided wherein the interference oligomer is an interfering RNA comprising a sequence having at least 95% sequence identity with GUAUGGACGGUGAAUGG (SEQ ID NO: 10).

In accordance with embodiment 10, the method of any one of embodiments 1-9 is provided wherein said interference oligomer is a phosphorodiamidate morpholino.

In accordance with embodiment 11, the method of any one of embodiments 1-10 is provided further comprising the step of transfecting said Tregs with a gene that encodes the FOXP3S isoform.

In accordance with embodiment 12, a method of treating cancer in a patient is provided wherein the method comprises the steps of increasing the intracellular concentrations of FOXP3S relative to FOXP3L in regulatory T cells (Tregs).

In accordance with embodiment 13, a method of treating cancer is provided wherein the increased relative concentration of FOXP3S is achieved by transfecting isolated Tregs in vitro with an interference oligomer that targets FOXP3 in said isolated Tregs; and reintroducing said transfected Tregs into said patient.

In accordance with embodiment 14, a method of treating cancer is provided wherein regulatory T cells (Tregs) are transfected in vivo with an interference oligomer in an amount effective to increase the intracellular concentrations of FOXP3S relative to FOXP3L in said Tregs.

In accordance with embodiment 15, a method of embodiments 12 or 14 is provided wherein the interference oligomer is a FOXP3S-promoting morpholino.

In accordance with embodiment 16, the method of any one of embodiments 1-15 is provided wherein the interference oligomer comprises a sequence of nucleobases selected from the group consisting of

CCATTCACCGTCCATACCTGGTG
CCTGCCCATTCACCGTCCATACC
TGCCCATTCACCGTCCATACCTG
GCCCATTCACCGTCCATACCTGG
TCCCTGCCCATTCACCGTCCATAC
TGCCCATTCACCGTCCATACCTGGT
and
TCCCTGCCCATTCACCGTCCATACCTGGTG.

In accordance with embodiment 17, the method of any one of embodiments 12-16 is provided wherein said interference oligomer is a phosphorodiamidate morpholino, optionally wherein the interference oligomer is an iRNA.

In accordance with embodiment 18, the method of any one of embodiments 1-17 is provided further comprising the step of transfecting said Tregs with a gene that encodes the FOXP3S isoform.

### EXAMPLE 1

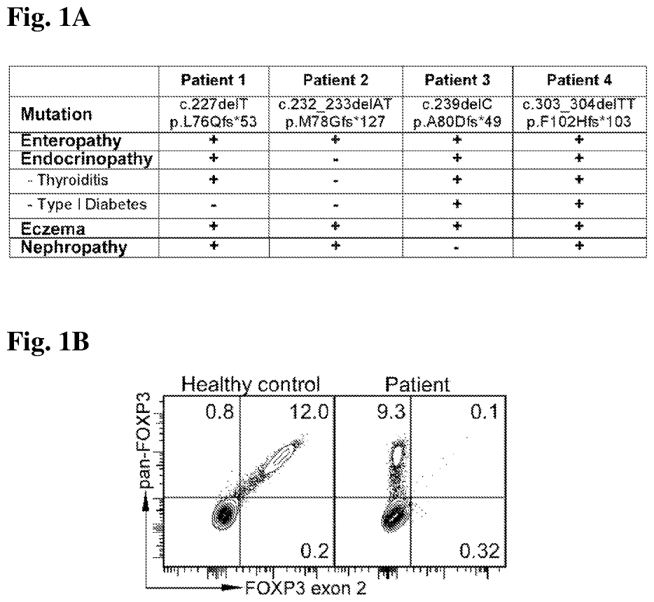

As disclosed herein 4 patients have been identified by the applicant as carrying deletion mutations in the exon 2 of FOXP3 (FIG. 1A). These mutations result in a frame shift in the exon 2-containing mRNA and thus altered and truncated the amino acid sequence of FOXP3L. However, the alternatively spliced FOXP3S isoform—excluding the mutated exon 2—is predicted to be intact. Staining peripheral blood mononuclear cells (PBMCs) from patient #3 with two anti-FOXP3 antibodies—pan-FOXP3 antibody (clone 259D) and exon 2-specific antibody (clone 150D)—demonstrated that 9.3% CD4⁺T cells in the patient stained positive for pan-FOXP3 but negative for exon 2-specific antibody indicating the expression of the FOXP3S isoform (FIG. 1B). In the healthy control, FOXP3S and FOXP3L were co-expressed through alternative splicing in a given Treg cell thus stained positively for both antibodies (FIG. 1B). Consistent with a recent study, our data demonstrated that without the FOXP3L isoform the FOXP3S isoform alone is insufficient to maintain self-tolerance, which leads to autoimmunity.

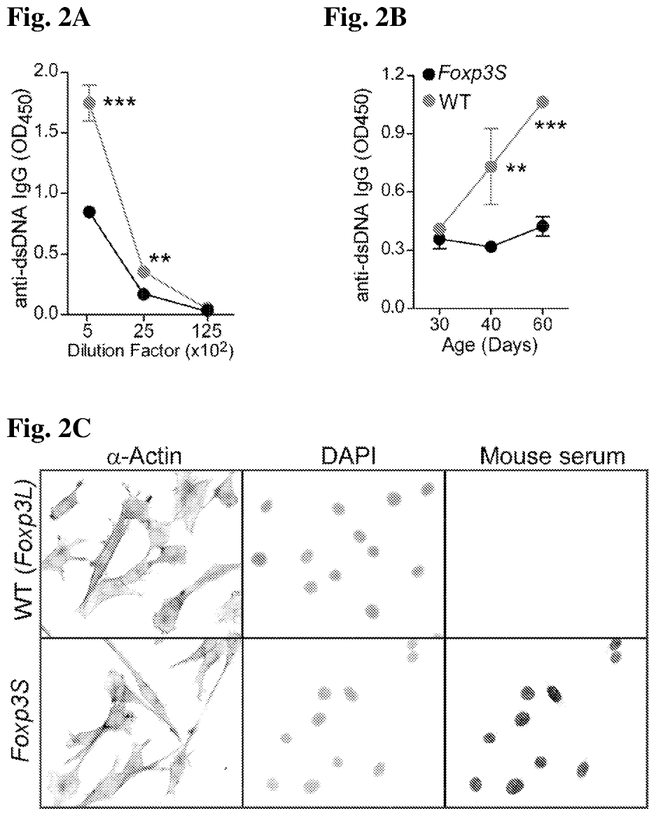

To study the functional difference of the two FOXP3 isoforms, we deleted exon 2 in the mouse Foxp3 gene (Foxp3S) that encodes the Foxp3 short isoform lacking exon 2, while leaving intact the intronic regulatory elements. Foxp3S mice were viable and morphologically normal with unaffected thymocyte and thymic Treg development. Compared with WT mice that only express the Foxp3L isoform, Foxp3S mice developed anti-dsDNA IgG (FIG. 2A) at the age of ˜40 days (FIG. 2B). Sera from two-month old Foxp3S mice strongly stained the nuclei of mouse 3T3 cells (FIG. 2C) indicating the existence of anti-nuclear autoantibody (ANA). These mice also developed eczema and nephropathy (data not shown) resembling the phenotypes in the patients who only express the FOXP3S isoform (FIG. 1A).

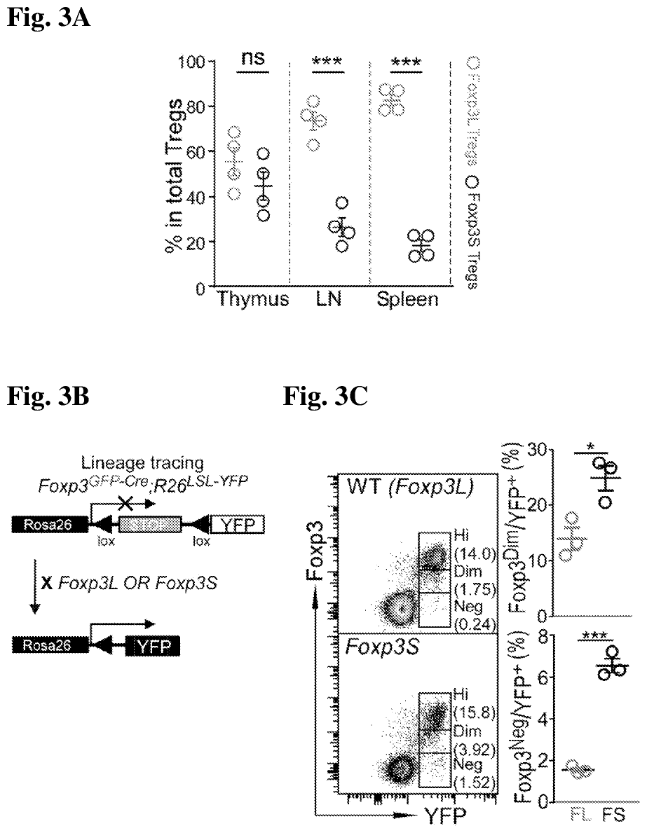

Since Foxp3S mice failed to maintain self-tolerance, we wondered whether Treg expressing only the Foxp3S isoform are defective in their ability to suppress effector T helpers. To our surprise, in an in vitro suppressive assay, Foxp3S Tregs and Foxp3L Tregs suppressed the proliferation of responder CD4⁺T cells equally well (data not shown). We examined Tregs in heterozygous Foxp3 exon 2 deletion (Foxp3S/L) female mice that had no signs of autoimmune diseases. Due to random X chromosome inactivation, these mice have two populations of Tregs, expressing either the Foxp3S isoform or the Foxp3L isoform, but not both. While ˜50% of Tregs in the thymus of Foxp3S/L female mice expressed the Foxp3S isoform, their frequencies were greatly reduced to ˜20% (FIG. 3A). Such reduced FOXP3S Treg frequency in periphery was also observed in a woman carrying one WT FOXP3 allele and the other FOXP3 allele encoding only the FOXP3S isoform due to deletion mutation (c.305delT) within the exon 2 region (published data). This heterozygous female had 17%, rather than 50%, circulating Tregs expressing only the FOXP3S isoform.

The much reduced frequency in the periphery (FIG. 3A) led us to hypothesize that Foxp3S Tregs are unstable. To test this hypothesis, we crossed Tg:Foxp3^{GFP-Cre}; Rosa26^{LSL-YFP} mice to Foxp3L (WT) or Foxp3S mice to generate lineage tracing mouse lines (FIG. 3B). We found that significantly more Foxp3S Tregs downregulated Foxp3 expression (YFP⁺Foxp3^{Dim} and YFP⁺Foxp3⁻) than Foxp3L Tregs (FIG. 3C), indicating instability of Foxp3S Tregs.

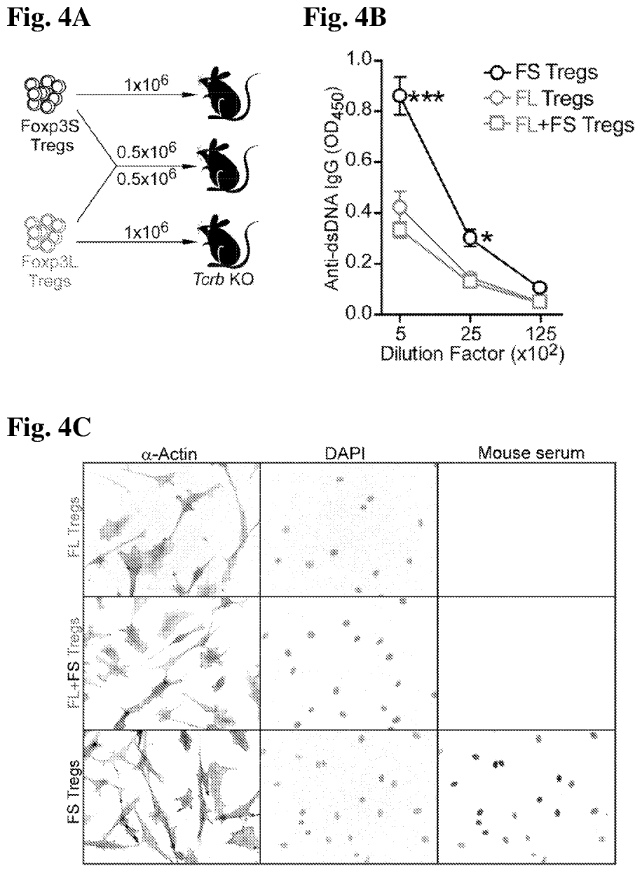

The fact that thymus-derived Tregs express self-reactive TCRs suggests the possibility that Foxp3S Tregs could become autoreactive effectors upon loss of Foxp3 expression, leading to autoimmunity. To test this hypothesis, we transferred purified Foxp3S and/or Foxp3L Tregs into Tcrb-deficient recipient mice as depicted in FIG. 4A. Three months after cell transfer, recipient mice that received Foxp3S Tregs developed anti-dsDNA IgG autoantibodies (FIG. 4B) and anti-nuclear autoantibodies (FIG. 4C). The recipient mice received Foxp3S Tregs had significantly increased follicular helper T cells and GC B cells than those that received Foxp3L Tregs or mixed Tregs. Since the recipient Tcrb-deficient mice do not have αβ cells, the Tfh cells seen in the mice that received Foxp3S Tregs are derived from adoptively transferred Tregs, indicating the ability of Foxp3S Tregs to transdifferentiate into self-reactive Tfh cells.

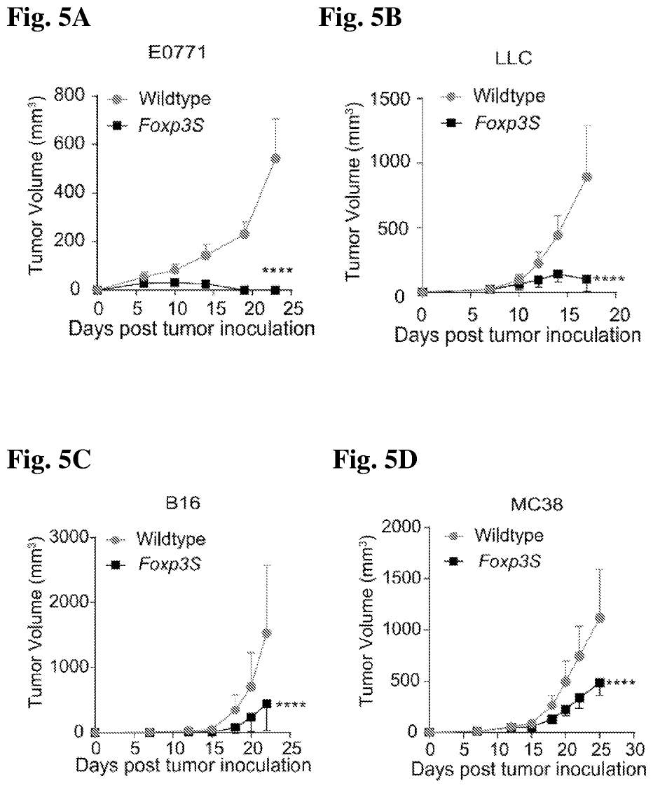

Our data that Foxp3S Tregs could differentiate into helper T cells and mediate autoreactive immune responses (FIGS. 4A-4C) led us to hypothesize that Foxp3S mice may confer resistance to tumor progression. 0.5×10⁶ EO771 breast cancer cells (a mouse triple negative breast tumor cell line) were orthotopically injected into the 4th mammary fat pad of WT and Foxp3S female mice. While all WT mice exhibited significant tumor growth (mean=200 mm³) 17 days after cancer cell injection, Foxp3S mice initially developed small masses ˜20-30 mm³ then regressed and disappeared (FIGS. 5A). This data strongly suggest that the Foxp3S isoform promotes anti-tumor immunity against the EO771 tumor growth. Similarly, we compared tumor growth of Lewis lung carcinoma (LLC) (FIG. 5B), B16 melanoma (FIG. 5C) and MC38 colon adenocarcinoma (FIG. 5D) and found all these tumors grew significant slower in Foxp3S mice than in WT mice.

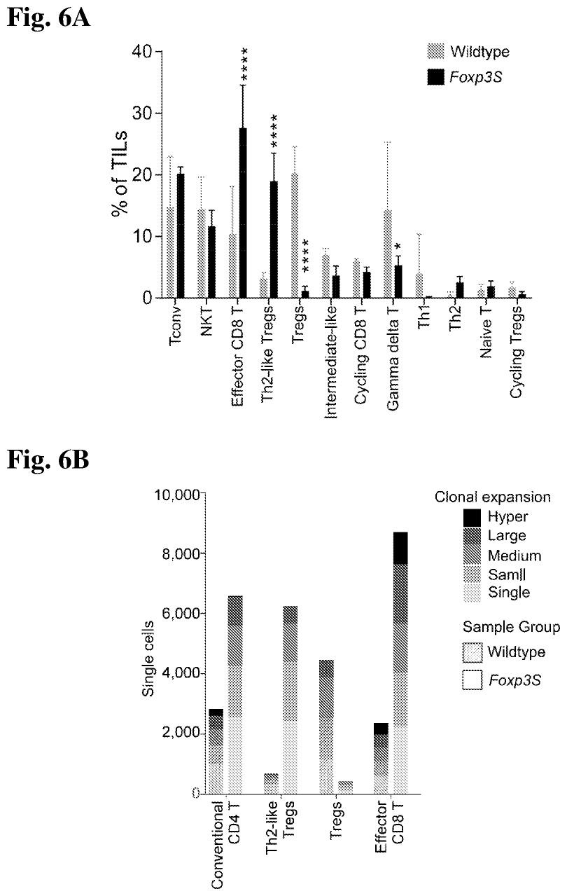

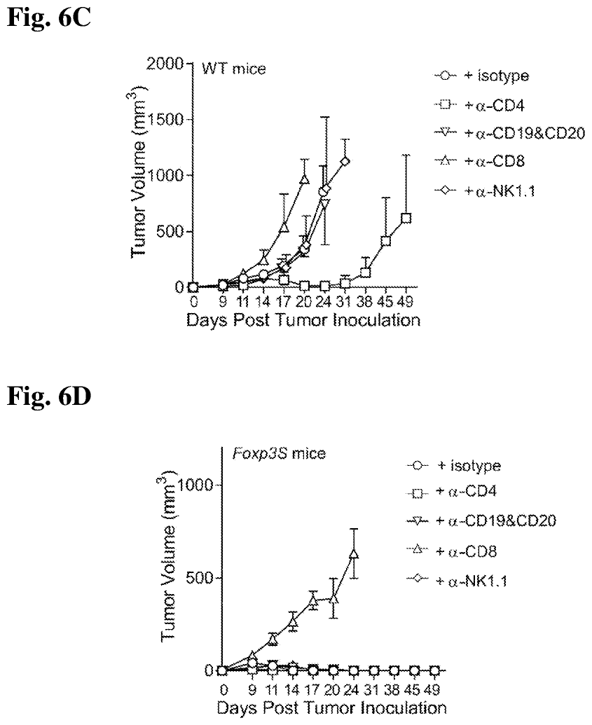

We performed single cell RNA sequencing to phenotype the immune cells infiltrating the EO771 tumors in WT and Foxp3S mice. We found that majority of the Foxp3S Tregs infiltrating the tumors showed T helper 2-like phenotypes while majority of the Foxp3L Tregs remained Treg phenotypes (FIG. 6A). There were also significant more effector CD8 cytotoxic cells infiltrating the tumors in Foxp3S mice than that in WT mice (FIG. 6A). T cell reporter (TCR) profiling analysis demonstrated greater clonal expansion of both CD4 and CD8 T cells in the EO771 TNBC tumors from Foxp3S mice than from WT mice (FIG. 6B). The enhanced antitumor immunity seen in Foxp3S mice is likely due to the ability of Foxp3S Tregs to provide help to CD8 cytotoxic T cells as antibody-mediated depletion of CD8 T cells restored EO771 TNBC growth in Foxp3S mice (FIGS. 6C & 6D)

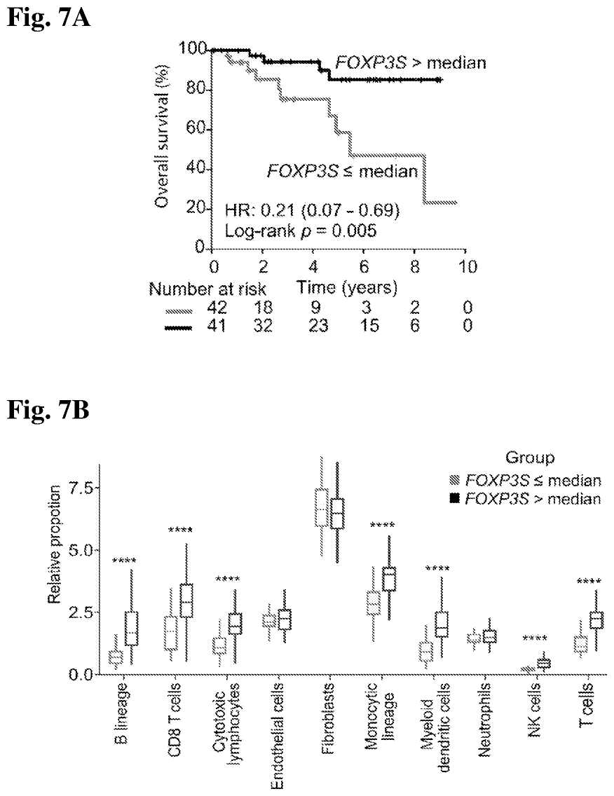

We next analyzed transcriptomic data sets of breast cancer tissues from The Cancer Genome Atlas (TCGA) to assess clinical relevance of FOXP3S isoform in human breast cancer. In triple negative breast cancer (TNBC) cases, FOXP3S expression in the breast cancer tissues is correlated with better overall survival (FIG. 7A). Using this data set, we estimated the compositions of the immune cells infiltrating the TNBC tissues with CIBERSORT—a versatile computational method. Consistent to our single cell RNA sequencing results (FIG. 6A), higher FOXP3S expression is positively correlated with increased CD8 and cytotoxic lymphocytes infiltrating the tumor tissues (FIG. 7B).

### EXAMPLE 2

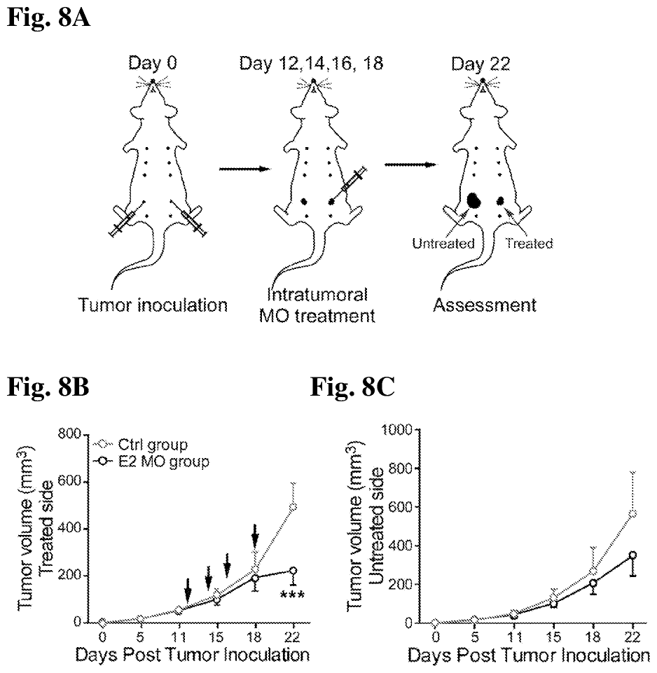

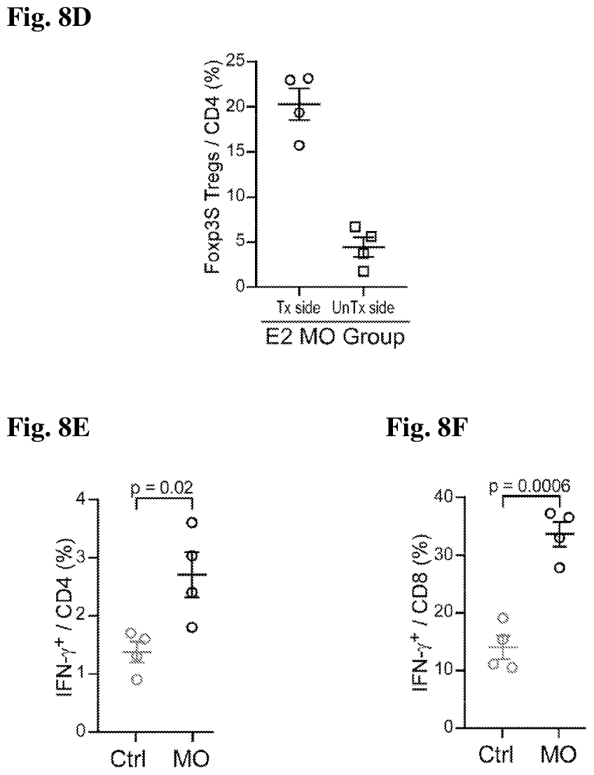

The efficacy of Foxp3 isoform shifting morpholino in triple negative breast cancer treatment is tested in mouse models. 1×10⁶ EO771 TNBC cells were orthotopically injected into the 4th mammary fat pad on both sides of the wild type mice. On days 12, 14, 16 and 18, 30 nmol/mouse of Foxp3 isoform shifting morpholino (E2 MO) or control MO was injected intratumorally into the tumors at the left side while leave the tumors at the right side as internal controls (FIG. 8A). Intratumoral injection of the E2 MO not only significantly suppressed the growth of treated tumors on the left-side (FIG. 8B) but also slowed down the growth of untreated tumors on the right-side (FIG. 8C). E2 MO treatment converted Foxp3L Tregs to Foxp3S Tregs. While no Foxp3S Tregs were seen in the tumors received control MO (data not shown), ˜30% Tregs in the E2 MO-injected tumors expressed the Foxp3S isoform (FIG. 8D). Furthermore, ˜6% Foxp3S Tregs were seen in tumors in the untreated tumors on the right-side of the same mice (FIG. 8D) Immunophenotyping of tumor infiltrating lymphocytes revealed that significantly higher proportion of CD4 (FIG. 8E) and CD8 (FIG. 8F) T cells in the E2 MO-injected tumors expressed IFN-γ.

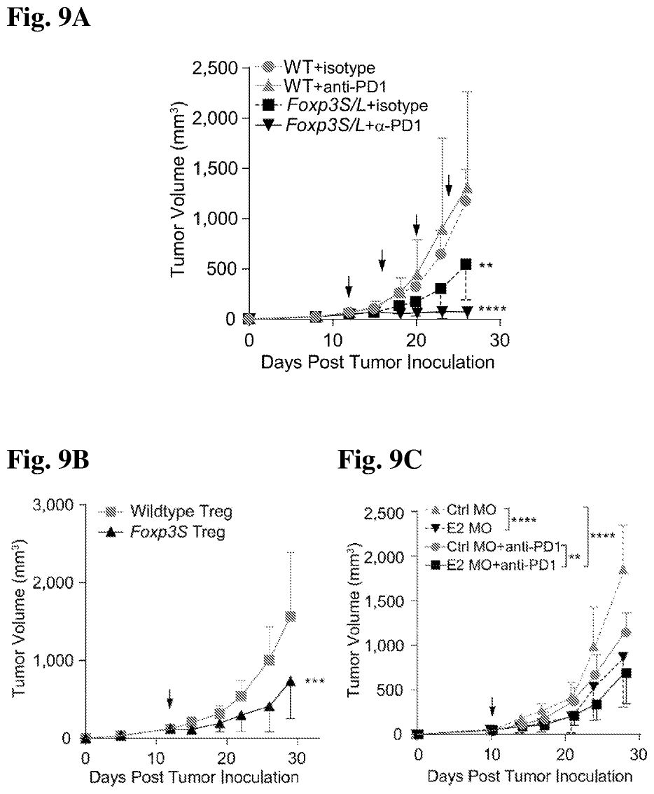

The results from the intratumoral injection of E2 MO (FIG. 8) suggest that the shift of Foxp3 expression to the Foxp3S isoform does not need to achieve 100% to promote antitumor immunity. To confirm this, we inoculated EO771 TNBC cells into the Foxp3S/L heterozygous mice that have 20-30% Tregs expressing Foxp3S isoform in the periphery (FIG. 3A). Indeed, tumors in Foxp3S/L mice grew significantly slower than in WT mice (FIG. 9A). Furthermore, administration of anti-PD-1 antibody (4 doses at 10 mg/kg between days 12 and 24) to Foxp3S/L mice harboring the tumor completely stopped tumor growth while treating tumor bearing WT mice with anti-PD-1 antibody had no effects on tumor growth (FIG. 9A).

To further test the ability of Foxp3S Tregs to boost antitumor immunity, Foxp3S Tregs and Foxp3LTregs were isolated from donor mice and injected intratumorally 2×10⁶ Tregs into the EO771 TNBC on day 12 post inoculation (FIG. 9B). The results demonstrated that Foxp3S Tregs alone is sufficient to enhance antitumor immunity and significantly slow down the tumor growth (FIG. 9B). This data suggest it is feasible to increase Foxp3S expression in Tregs in vitro and reintroduce the cells back into mice to boost antitumor immunity. We then isolated total T cells from the draining lymph nodes of tumor-bearing WT mice and expanded in vitro in the presence of isoform shifting E2 MO or control MO (1 μM) for 7 days. 2×10⁶ expanded T cells were injected into the tumors grown in WT mice for 10 days. T cells expanded with E2 MO significantly reduced tumor growth and combination with anti-PD-1 treatment further enhanced the efficacy (FIG. 9C).

### EXAMPLE 3

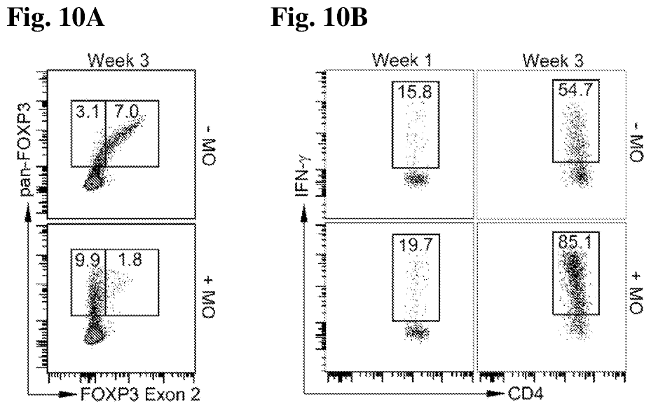

Peripheral blood mononuclear cells were obtained from healthy donors and cultured in vitro with 1 μM isoform-shifting morpholino (E2 MO) for 3 weeks. Flow cytometry analysis demonstrated that majority of the Tregs expressed only FOXP3S isoform (positive for pan-FOXP3 antibody staining but negative for FOXP3 exon 2-specific antibody staining) while Tregs without MO treatment expressed FOXP3L (positive for FOXP3 exon 2-specific antibody staining) (FIG. 10A). The MO treated Tregs down-regulated inhibitory molecules but up-regulated co-stimulatory molecules normally expressed on helper T cells (data not shown). Furthermore, MO treated Tregs expressed higher effector cytokine IFN-γ (FIG. 10B). Therefore, we identified morpholinos that are highly effective to shift human FOXP3 expression to the FOXP3S isoform and such isoform shifting results in human regulatory T cells (Tregs) to up-regulate co-stimulatory molecules and effector cytokines.

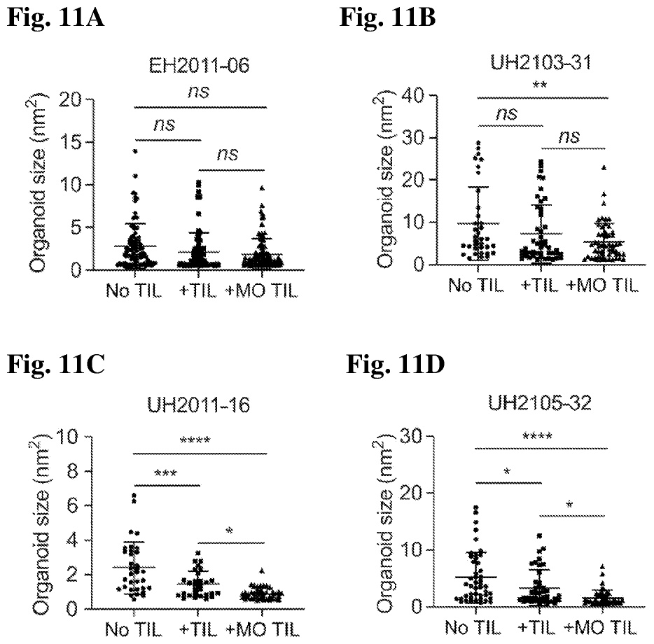

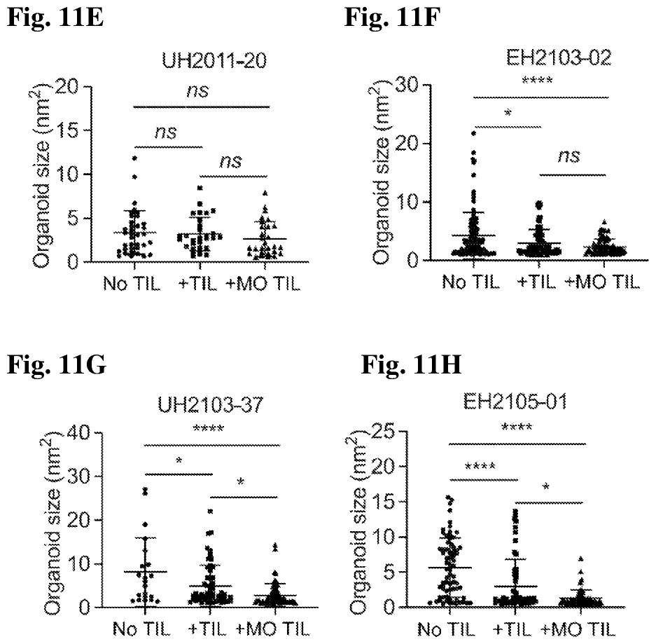

We next tested whether tumor infiltrating lymphocytes expanded in the presence of E2 MO have enhanced killing of autologous tumor cells. Tumor resections from breast cancer and colon cancer patients were obtained. Tumor organoids were generated from tumor cells from the resections and tumor infiltrating lymphocytes (TIL) were expanded in vitro in the presence or absence of E2 MO. After 7 days expansion, TILs were co-cultured with autologous tumor organoids and the size of the organoids was measured. 3 out of 4 E2 MO expanded TILs from breast cancer patients (FIG. 11A-11D) as well as colon cancer patients (FIG. 11E-11H) showed significantly enhanced killing of their corresponding organoids.

1. A method for altering regulatory T cells (Tregs) activity, said method comprising the step of modifying intracellular concentrations of FOXP3 isoforms FOXP3L and FOXP3S in said Tregs, wherein the amount of the FOXP3S isoform is increased relative to the FOXP3L isoform.

2. The method of claim 1 wherein the expression of the FOXP3L is inhibited relative to FOXP3S by transfecting Tregs with an interference oligomer that targets FOXP3L, optionally by targeting exon 2 of FOXP3.

3. The method of claim 2 wherein the expression of the FOXP3S is enhanced relative to FOXP3L by transfecting Tregs with a nucleic acid encoding for the FOXP3S isoform.

4. The method of claim 3 wherein said Tregs are transfected with an amount of said interference oligomer sufficient to induce the isolated Tregs to transdifferentiate into helper-like T cells.

5. The method of claim 4 wherein the relative expression of the FOXP3L isoform is decreased by transfecting said Tregs with a FOXP3L targeting interference oligomer comprising
i) a nucleobase sequence having at least 85% sequence identity with SEQ ID NO: 1 or SEQ ID NO: 12, or
ii) at least a contiguous 10 nucleobase fragment of SEQ ID NO: 1 or SEQ ID NO: 12; or
iii) a complement of i) or ii).

6. The method of claim 5 wherein said targeting interference oligomer comprises the nucleobase sequence of TGCCCATTCACCGTCCATACCTGGT (SEQ ID NO: 8), or a complement thereof.

7. The method of claim 4 wherein the relative expression of the FOXP3L isoform is decreased by transfecting said Tregs with a FOXP3L targeting interference oligomer that specifically binds to a sequence comprising CCATTCACCGTCCATAC (SEQ ID NO: 2) or CCAUUCACCGUCCAUAC (SEQ ID NO: 9).

8. The method of claim 7 wherein the interference oligomer specifically binds to a sequence selected from the group consisting of
CCATTCACCGTCCATACCTGGTG (SEQ ID NO: 2),
CCTGCCCATTCACCGTCCATACC (SEQ ID NO: 4),
TGCCCATTCACCGTCCATACCTG (SEQ ID NO: 5),
GCCCATTCACCGTCCATACCTGG (SEQ ID NO: 6),
TCCCTGCCCATTCACCGTCCATAC (SEQ ID NO: 7)
TGCCCATTCACCGTCCATACCTGGT (SEQ ID NO: 8) and
TCCCTGCCCATTCACCGTCCATACCTGGTG (SEQ ID NO: 1) or its RNA counterpart.

9. The method of claim 8 wherein the interference oligomer is an interfering RNA comprising a sequence having at least 95% sequence identity with GUAUGGACGGUGAAUGG (SEQ ID NO: 10).

10. (canceled)

11. (canceled)

12. A method of treating cancer in a patient comprising the steps of increasing the intracellular concentrations of FOXP3S relative to FOXP3L in regulatory T cells (Tregs).

13. The method of claim 12 wherein the increased relative concentration of FOXP3S is achieved by
transfecting isolated Tregs in vitro with an interference oligomer that targets FOXP3 in said isolated Tregs; and
reintroducing said transfected Tregs into said patient.

14. The method of claim 12 wherein regulatory T cells (Tregs) are transfected in vivo with an interference oligomer in an amount effective to increase the intracellular concentrations of FOXP3S relative to FOXP3L in said Tregs.

15. (canceled)

16. The method of claim 12 wherein the expression of FOXP3L is decreased by transfecting Tregs with an interference oligomer that targets FOXP3 exon 2 and comprises a sequence that binds to
i) nucleobases CTGCCCACACTGCCCCTAGTCATGGTGGCACCCTCCGGGGCACGGCTGGGCCCCTTG CCCCACTTACAGGCACTCCTCCAGGACAGGCCACATTTCATGCACCAG (SEQ ID NO: 12) or a complete complement thereof, or
ii) a contiguous 10, 15, 17, 20 or 25 bp or longer fragment sequence of SEQ ID NO: 12 or its complement thereof.

17. The method of claim 16 wherein the interference oligomer is an interference RNA selected from the group consisting of a small interfering RNA (siRNA), and microRNA (miRNA).

18. (canceled)

19. The method of claim 16 wherein the interference oligomer comprises a sequence of nucleobases selected from the group consisting of
(SEQ ID NO: 2)
CCATTCACCGTCCATACCTGGTG,
(SEQ ID NO: 4)
CCTGCCCATTCACCGTCCATACC,
(SEQ ID NO: 5)
TGCCCATTCACCGTCCATACCTG,
(SEQ ID NO: 6)
GCCCATTCACCGTCCATACCTGG,
(SEQ ID NO: 7)
TCCCTGCCCATTCACCGTCCATAC,
(SEQ ID NO: 8)
TGCCCATTCACCGTCCATACCTGGT,
and
(SEQ ID NO: 1)
TCCCTGCCCATTCACCGTCCATACCTGGTG.

20. The method of claim 19 wherein said interference oligomer is a phosphorodiamidate morpholino or an interference RNA.

21. (canceled)

22. The method of claim 19 further comprising the step of transfecting said Tregs with a gene that encodes the FOXP3S isoform.

23. The method of claim 19 further comprising the step of administering to said patient an immune checkpoint blockade PD-1 antibody in combination of FOXP3S-promoting morpholino in the primary or metastatic TNBC models.

24. A method of inducing regulatory T cells (Tregs) to transdifferentiate into tumor-reactive helper-like T cells, said method comprising transfecting said Tregs with a FOXP3S-promoting interference oligomer in an amount effective to enhanced FOXP3S expression in said Tregs.

25. The method of claim 24 wherein regulatory T cells (Tregs) are transfected in vivo with an interference oligonucleotide that binds to exon 2 of FOXP3L, wherein said transfection is via tissue nanotransfection.

26. -28. (canceled)
